(19) United States
(12) Patent Application Publication
(10) Pub. No.: US 2018/0243006 A1
(43) Pub. Date: Aug. 30, 2018

(54) EXTERNAL FIXATION LOCKING DEVICE

(71) Applicant: Ke MA, Beijing (CN)

(72) Inventor: Ke MA, Beijing (CN)

(21) Appl. No.: 15/753,503

(22) PCT Filed: Jan. 22, 2016
(86) PCT No.: PCT/CN2016/000047

§ 371 Date: Feb. 19, 2018

(30) Foreign Application Priority Data
Oct. 27, 2015 (CN) 201510707497.5

(51) Int. Cl. A61B 17/60

(52) U.S. Cl. CPC A61B 17/60; A61B 2017/00477

(57) ABSTRACT

An external fixation locking device comprises a bolt (1), a spring (2), a spherical washer (3), a top piece (4), a middle piece (5) and a tube locking bottom piece (6), wherein the top piece (4) is a tube locking top piece (41) or a pin locking top piece (42), wherein the spring (2), the spherical washer (3), the top piece (4), the middle piece (5) and the tube locking bottom piece (6) are successively threaded onto the screw rod of the bolt (1), wherein the axes of the spring (2), the spherical washer (3), the top piece (4), the middle piece (5) and the bottom piece (6) coincide with the axis of the bolt (1).

This application is the U.S. national phase of International Application No. PCT/CN2016/000047 filed on 22 Jan. 2016 which designated the U.S. and claims priority to Chinese Application No. CN201510707497.5 filed on 27 Oct. 2015, the entire contents of each of which are hereby incorporated by reference.

The present application claims the priority of the Chinese patent application entitled “Locking Device for External Fixation” filed on Oct. 27, 2015 with the application number of 201510707497.5. The entire content thereof is hereby incorporated by reference.

### TECHNICAL FIELD

The present invention relates to the field of a medical device, and particularly relates to a locking device for external fixation.

### BACKGROUND ART

The existing pin (tube) locking clamp for external fixation comprises a pin locking clamp and a tube locking clamp which are connected to a fastening bolt in series, and the operational principles thereof are as follows: loosening the fastening bolt to a preset target distance, so that a rod and a pin can be pressed into a rod groove and a pin groove of the pin (tube) locking clamp respectively, tightening the fastening bolt, so that the rod and pin are maintained in a position relation required by the operation, and then performing the operation of fixation. However, the existing pin (tube) locking clamp has the following deficiencies:

1. As the pin groove is applicable for various pins with different diameters used in a operation process, so while pins with different diameters being pressed in, the pin locking top piece tilts with different angles, which results in that the fastening bolt is partially unevenly loaded in a clamped state, which may cause that the fastening bolt is slightly bent, as a result, the possibility of loosening of the pin and the rod increases;

2. When the existing pin (tube) locking clamp is used in an operation process, the fixing rod is first placed, then the pin is threaded, and afterwards a fastening bolt is tightened. Because pin is threaded before the fastening bolt is tightened, adequate distances to the pin locking middle piece and the tube locking middle piece have to be left, so as to ensure that the steel pin can be inserted. However, at this moment, when the pin (tube) locking clamp is locked, the position of the pin or rod may be changed. Especially when the pin locking clamp is locked, it results in an increased internal stress of the steel pin, so that the risk that the fracture sections of the fractured bone segments connected to the pin is subjected to an angular displacement in the back and forth direction or lateral displacement increases. Therefore, in the operation process, when a pin is threaded by a physician, the requirement for selecting a pin threading point is stringent. However, no matter how a pin threading point is selected, it is impossible to avoid stress deformation of the pin during the locking, which directly results in a large probability of a post-surgery dispute over the medical quality;

3. As the top surfaces of the pin locking middle piece and the tube locking middle piece are concave and convex tooth-shaped surfaces, the angular positions of the rod and the pin in the operation process is limited by the concave and convex tooth-shaped surfaces, which results in internal stress of the pin and restriction of the pin threading angle.

### CONTENT OF THE INVENTION

The object of the present invention is to provide an external fixation locking device, so as to solve the aforementioned problems present in the prior art.

In order to realize the aforementioned object, the present invention discloses a locking device for external fixation, the locking device comprises: a bolt, a spring, a spherical washer, a top piece, a middle piece and a tube locking bottom piece, wherein the top piece is a tube locking top piece or a pin locking top piece, wherein the spring, the spherical washer, the top piece, the middle piece and the tube locking bottom piece are successively threaded onto the screw rod of the bolt, wherein the axes of the spring, the spherical washer, the top piece, the middle piece and the bottom piece coincide with the axis of the bolt, and the spring, the spherical washer, the top piece, the middle piece and the tube locking bottom piece are sequentially fixed by connecting the bolt to the tube locking bottom piece, wherein the spring is disposed adjacent to the head of the bolt.

Preferably, the spherical washer is a hemisphere with parallel upper and lower surfaces, within which hemisphere a first through hole matched with the screw rod and a second through hole matched with the spring are provided, wherein the first through hole and the second through hole form together a step-shaped through hole and their axes coincide with each other, wherein the diameter of the first through hole is less than that of the second through hole and the second through hole communicates with the first through hole.

More preferably, one end of the spring is adjacent to the bottom surface of the head of the bolt, and the other end thereof is supported on an annular shoulder formed by the second through hole and the first through hole.

More preferably, a spherical washer groove matched with the spherical washer is provided on the top surface of the top piece and the spherical washer is disposed within the spherical washer groove; and the lower surface of the spherical washer is in contact with the top surface of the top piece, the upper surface of the spherical washer has a diameter larger than that of the lower surface of the spherical washer.

Preferably, a pin groove provided on the bottom surface of the tube locking top piece, a second tube groove provided in the bottom surface of the pin locking top piece and a first tube groove provided in the top surface of the tube locking bottom piece are all disposed towards the middle piece.

Preferably, the middle piece is a circular ring centrally provided with a through hole matched with the screw rod, and the upper surface and the lower surface of the circular ring are both provided with a knurled section.

More preferably, a first projection is provided on the bottom surface of the top piece where no groove is provided, wherein the first projection is perpendicular to the upper surface of the circular ring and the surface of the first projection in contact with the upper surface of the circular ring is planar.

More preferably, a second projection is provided on the top surface of the tube locking bottom piece where the first tube groove is not arranged, wherein the second projection is perpendicular to the lower surface of the circular ring, and wherein the surface of the second projection in contact with the lower surface of the circular ring is planar.

More preferably, the upper surface and the lower surface of the middle piece are both provided with an inner recess, which lies in a plane that is parallel to a plane in which the knurled section is situated.

Preferably, the screw rod includes an upper rod portion with a smooth outer surface and a lower rod portion with a threaded outer surface, and the sidewall of the through hole centrally disposed in the tube locking bottom piece is provided with a thread matched with the screw rod.

The advantageous effects of the present invention are as follows:

1. The locking device for external fixation according to the present invention is provided with a spherical washer, so that the bending deformation of the bolt is effectively reduced and at the same time, during and after an operation, the fastening piece is prevented from loosening and thus the medical staff doesn't need to re-tighten the bolt.

2. According to the present invention, the spring of the locking device for external fixation is disposed in the spherical washer, therefore the displacement of the pin due to the mentioned problem upon threading the pin during an operation is avoided, and the deformation of the steel pin caused by internal stress due to the displacement of the steel pin is substantially reduced, which means that the requirement to be met while threading the pin is less stringent and the probability of post-surgery dispute over the medical quality is lower, i.e. the expected effect of the operational treatment can be ensured.

3. The locking device for external fixation according to the present invention is provided with only one middle piece, which allows that a pin threading angle is not restricted and an internal stress is reduced due to the use of a multi-face knurled and circular configuration.

### BRIEF DESCRIPTION OF THE DRAWINGS

FIG. 1 is a schematic view of the structure of the locking device for external fixation in which the top piece is a tube locking top piece;

FIG. 2 is a schematic view of the structure of the locking device for external fixation in which the top piece is a pin locking top piece;

FIG. 3 is a schematic longitudinal section view of the spherical washer in FIG. 1 and FIG. 2;

FIG. 4 is a schematic view of the structure of the top piece of the locking device for external fixation;

FIG. 5 is a schematic longitudinal section view of the middle piece in FIG. 1 and FIG. 2;

FIG. 6 is a schematic top-view of the middle piece in FIG. 1 and FIG. 2;

FIG. 7 is a schematic longitudinal section view of the tube locking bottom piece in FIG. 1 and FIG. 2.

### EMBODIMENTS

In order to make the object, technical solution and advantages of the present invention clear and explicit, the present invention will be further described in detail in combination with the drawings as follows. It should be understood that the embodiments described here are only used for explaining the present invention, rather than for limiting the present invention.

### EXAMPLES

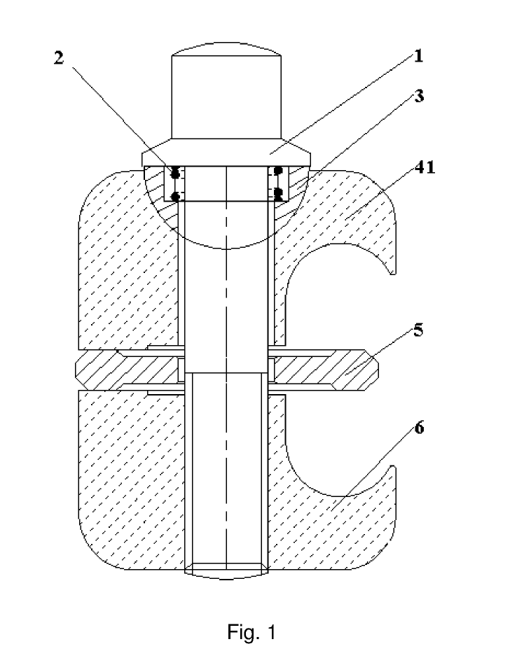

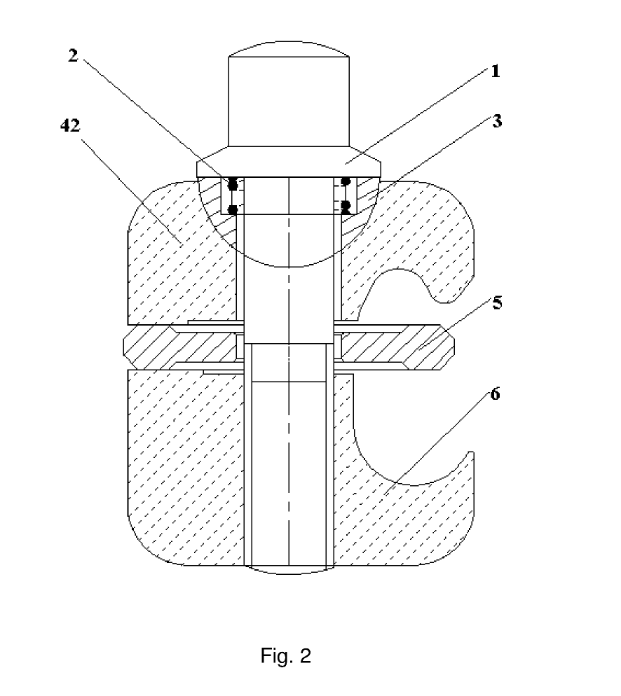

FIG. 1 and FIG. 2 show an embodiment of the locking device for external fixation according to the present invention, wherein the locking device comprises: a bolt 1, a spring 2, a spherical washer 3, a top piece, a middle piece 5 and a tube locking bottom piece 6. The top piece is a tube locking top piece 41 or a pin locking top piece 42. The spring 2, the spherical washer 3, the top piece 4, the middle piece 5 and the tube locking bottom piece 6 are successively threaded onto the screw rod of the bolt 1, wherein the axes of the spring 2, the spherical washer 3, the top piece 4, the middle piece 5 and the bottom piece 6 coincide with the axis of the bolt 1. The spring 2, the spherical washer 3, the top piece, the middle piece 5 and the tube locking bottom piece 6 are sequentially fixed by connecting the screw rod 1 with the tube locking bottom piece 6, wherein the spring 2 is disposed adjacent to the head of the bolt 1. A more detailed explanation is as follows:

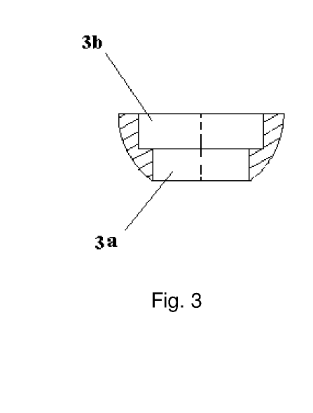

(i) As shown in FIG. 3, the spherical washer 3 is a hemisphere with parallel upper and lower surfaces, within which hemisphere a first through hole 3a matched with the screw rod and a second through hole 3b matched with the spring are provided. The first through hole 3a and the second through hole 3b forms together a step-shaped through hole and their axes coincide with each other, wherein the diameter of the first through hole 3a is less than that of the second through hole 3b. The second through hole 3b communicates with the first through hole 3a, wherein the first through hole 3a is disposed in the lower portion of the spherical washer 3, while the second through hole 3b is disposed in the upper portion of the spherical washer 3.

One end of the spring 2 contacts with the bottom surface of the head of the bolt 1, and the other end thereof is supported on an annular shoulder formed by the second through hole 3b and the first through hole 3a.

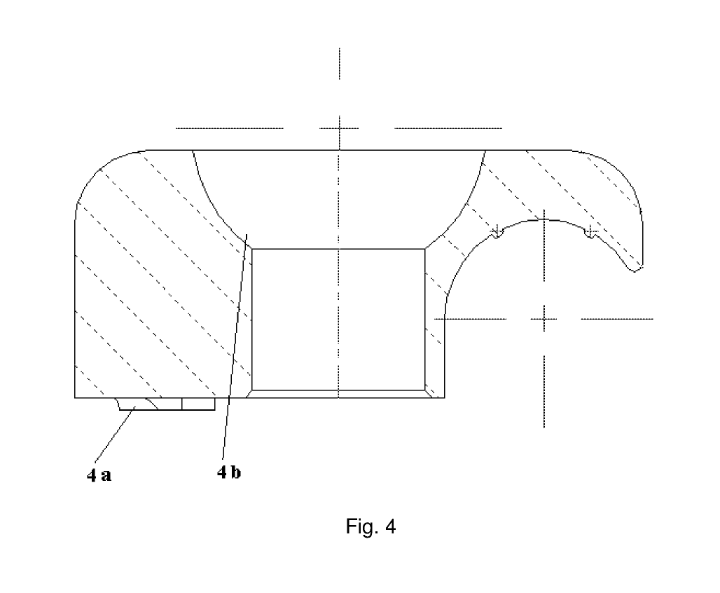

(ii) As shown in FIG. 4, a spherical washer groove 4b matched with the spherical washer 3 is provided on the top surface of the top piece, the spherical washer 3 is disposed within the spherical washer groove 4b; a lower surface of the spherical washer 3 is in contact with the bottom surface of the spherical washer groove 4b, the upper surface of the spherical washer has a diameter larger than that of the lower surface of the spherical washer, and the spherical washer 3 has a height that is greater than the depth of the spherical washer groove 4b.

(iii) As shown in FIG. 4, a second tube groove provided in the bottom surface of the tube locking top piece 41, a pin groove provided on the bottom surface of the pin locking top piece 42 and a first tube groove provided in the top surface of the tube locking bottom piece 6 are all disposed towards the middle piece, and none of the second tube groove, the pin groove and the first tube groove intersects with the axis of the associated components, i.e. the tube locking top piece 41, the pin locking top piece 42 and the tube locking bottom piece 6, in which said grooves are situated.

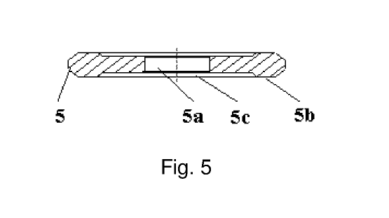

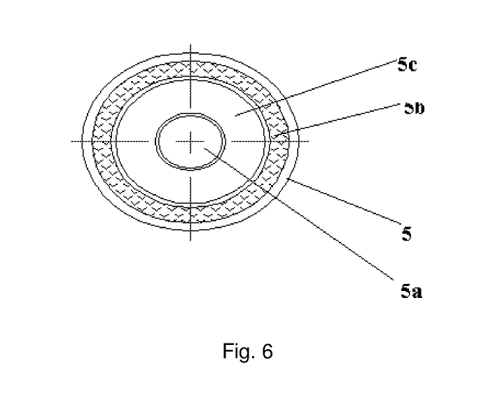

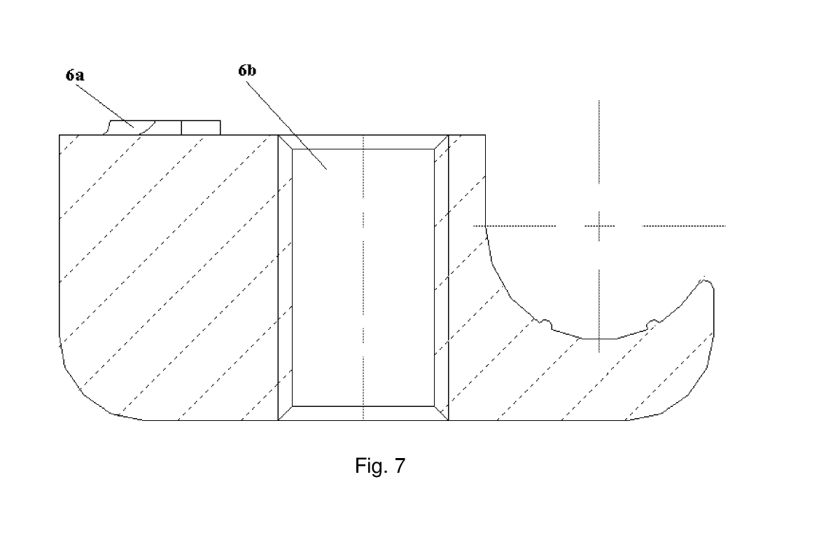

(iv) As shown in FIGS. 4, 5, 6 and 7, the middle piece 5 is a circular ring centrally provided with a through hole 5a matched with the screw rod, and the upper surface and the lower surface of the circular ring are both provided with a knurled section 5b.

A first projection 4a is provided on the bottom surface of the top piece where no groove is provided, wherein the first projection 4a is perpendicular to the upper surface of the circular ring and the surface of the first projection 4a in contact with the upper surface of the circular ring is planar.

A second projection 6a is provided on the top surface of the tube locking bottom piece, which is perpendicular to the lower surface of the circular ring, wherein the surface of the second projection 6a in contact with the lower surface of the circular ring is planar.

The upper surface and the lower surface of the middle piece 5 are both provided with an inner recess 5c which lies in a plane that is parallel to a plane in which the knurled section 5b is situated.

(v) The screw rod 1 includes an upper rod portion with a smooth outer surface and a lower rod portion with a threaded outer surface, and the sidewall of the through hole 6b centrally disposed in the tube locking bottom piece 6 is provided with a thread matched with the screw rod 1.

The bolt in the present application is a fastening bolt for securing the entire device. The spring is used for a preloading effect. In an operation process, pins with different diameters might be used, so the pin locking top piece may tilt up and down with different angles. To deal with the situation, the spherical washer according to the present application rotates while the bolt being tightened, so that the bolt and the pin locking top piece remain vertical to each other, so that the bolt is prevented from bending. The pin locking top piece is used for fixing the steel pin in cooperation with the middle piece. The middle piece is used in cooperation with the pin locking top piece and the tube locking bottom piece to fix the steel pin and the rod. The tube locking bottom piece is used in cooperation with the middle piece to fix the rod.

In the present application, the top surface of the top piece is provided with a spherical washer recess mated with the spherical washer to enable the spherical washer to rotate under the effect of a force, so that the upper surface of the spherical washer remains perpendicular to the screw rod of the fastening bolt. A spring is arranged on the spherical washer. Under the effect of the spring force, the top piece is permanently in contact with the middle piece, and the middle piece is permanently in contact with the tube locking bottom piece. Because of the preloading force of the spring, when the rod and the pin are clamped, tightening the bolt causes no change of the distance between the horizontal planes, in which the rod and the pin lie. The upper and lower surfaces of the middle piece are knurled, which increases the friction force on one hand, and can keep the rod and the pin in any angle position without any limitation on the other hand.

In an operation process, the bolt is loosened to a certain extent, so that an effective distance required for clamping a rod and a pin can be reached.

Firstly, no matter which one of the rod and the pin is clamped first, under the effect of the spring force, the middle piece is always in contact with the top piece and the pin locking bottom piece, i.e. the distance therebetween is not changed. When the bolt is tightened, the bolt moves downwards, and the top piece, the middle piece and the pin locking bottom piece are not moved, so that no displacement of the rod and the pin is caused. Secondly, in the operation process, as pins with different sizes are selected, the pin locking top piece correspondingly tilts up and down with different angles, therefore, traditionally, the bottom surface of the bolt head contacts the pin locking top piece locally. According to the present invention, the problem of local contact between the bottom surface of the bolt head and the pin locking top piece can be avoided by providing the spherical washer. By means of spherical rotation, the bolt and the spherical washer can always remain vertical to each other, and the rod and the pin can be clamped through force transmission. Finally, the middle piece is designed as a circular ring with knurled end faces, and the top piece can rotate about the bolt, so that the pin clamping groove of the pin locking top piece can be adapted to the angle of the threaded bone pin. Thus, according to the present invention, there is no limitation on the angle of pins in practice.

With the above technical solution disclosed by the present invention, the following advantageous effects are obtained:

1. The locking device for external fixation according to the present invention is provided with a spherical washer, so that the bending deformation of the bolt is effectively reduced and at the same time, during and after an operation, the fastening piece is prevented from loosening and thus the medical staff doesn't need re-tighten the bolt.

2. According to the present invention, the spring of the locking device for external fixation is disposed in the spherical washer, therefore the displacement of the pin due to the mentioned problem upon threading the pin during an operation is avoided, and the deformation of the steel pin caused by internal stress due to the displacement of the steel pin is substantially reduced, which means that the requirement to be met while threading the pin is less stringent and the probability of post-surgery dispute over the medical quality is lower, i.e. the expected effect of the operational treatment can be ensured.

3. The locking device for external fixation according to the present invention is provided with only one middle piece, which allows that a pin threading angle is not restricted and an internal stress is reduced due to the use of a multi-face knurled and circular configuration.

The aforementioned only pertains to preferred embodiments of the present invention. It should be set forth that, for a person skilled in the art, on the premise of not departing away from the principles of the present invention, several improvements and modifications may also be made to the present invention, and such modifications and decorations should also be deemed as falling into the protection scope of the present invention.

What is claimed is:

1. A locking device for external fixation, characterized in that, the locking device comprises: a bolt, a spring, a spherical washer, a top piece, a middle piece and a tube locking bottom piece, wherein the top piece is a tube locking top piece or a pin locking top piece,
wherein the spring, the spherical washer, the top piece, the middle piece and the tube locking bottom piece are successively threaded onto the screw rod of the bolt, wherein the axes of the spring, the spherical washer, the top piece, the middle piece and the bottom piece coincide with the axis of the bolt, and the spring, the spherical washer, the top piece, the middle piece and the tube locking bottom piece are sequentially fixed by connecting the screw rod to the tube locking bottom piece, wherein the spring is disposed adjacent to the head of the bolt.

2. The device according to claim 1, characterized in that, the spherical washer is a hemisphere with parallel upper and lower surfaces, within which hemisphere a first through hole matched with the screw rod and a second through hole matched with the spring are provided, wherein the first through hole and the second through hole form together a step-shaped through hole and their axes coincide with each other, wherein the diameter of the first through hole is less than that of the second through hole and the second through hole communicates with the first through hole.

3. The device according to claim 2, characterized in that, one end of the spring is adjacent to the bottom surface of the head of the bolt, and the other end thereof is supported on an annular shoulder formed by the second through hole and the first through hole.

4. The device according to claim 2, characterized in that, a spherical washer groove matched with the spherical washer is provided on the top surface of the top piece and the spherical washer is disposed within the spherical washer groove; the lower surface of the spherical washer is in contact with the top surface of top piece, the upper surface of the spherical washer has a diameter larger than that of the lower surface of the spherical washer.

5. The device accord ng to claim 1, characterized in that, a second tube groove provided in the bottom surface of the tube locking top piece, a pin groove provided on the bottom surface of the pin locking top piece and a first tube groove provided in the top surface of the tube locking bottom piece are all disposed towards the middle piece.

6. The device according to claim 1, characterized in that, the middle piece is a circular ring centrally provided with a through hole matched with the screw rod, and the upper surface and the lower surface of the circular ring are both provided with a knurled section.

7. The device according to claim 6, characterized in that, a first projection is provided on the bottom surface of the top piece where no groove is provided, wherein the first projection is perpendicular to the upper surface of the circular ring and the surface of the first projection in contact with the upper surface of the circular ring is planar.

8. The device according to claim 6, characterized in that, a second projection is provided on the top surface of the tube locking bottom piece where the first tube groove is not arranged, wherein the second projection is perpendicular to the lower surface of the circular ring, and wherein the surface of the second projection in contact with the lower surface of the circular ring is planar.

9. The device according to claim 6, characterized in that, the upper surface and the lower surface of the middle piece are both provided with an inner recess, which lies in a plane that is parallel to a plane in which the knurled section is situated.

10. The device according to claim 1, characterized in that, the screw rod includes an upper rod portion with a smooth outer surface and a lower rod portion with a threaded outer surface, and the sidewall of the through hole centrally disposed in the tube locking bottom piece is provided with a thread matched with the screw rod.
